SEQUENCE LISTING (4 sequences)

SEQ ID NO: 1 - DNA, 72 bp, Homo sapiens
  source 1..72: mol_type = genomic DNA
ggtcgggtgaggcgcaaaaggataaaaagccggtggaagcggagctgagcagatccgagccgggctggctgc

SEQ ID NO: 2 - DNA, 178 bp, synthetic construct
  source 1..178: mol_type = other DNA
aagcttctcgaggaggcgaaacttctggaatattcccgaaacgaaacttctggaatattcccgaaacgaaacttctggaatattcccgaaagttggtcgggtgaggcgcaaaaggataaaaagccggtggaagcggagctgagcagatccgagccgggctggctgcaagcttgaattc

SEQ ID NO: 3 - DNA, 328 bp, synthetic construct
  source 1..328: mol_type = other DNA
aagcttctcgaggaggcgaaacttctggaatattcccgaaacgaaacttctggaatattcccgaaacgaaacttctggaatattcccgaaacgaaacttctggaatattcccgaaacgaaacttctggaatattcccgaaacgaaacttctggaatattcccgaaacgaaacttctggaatattcccgaaacgaaacttctggaatattcccgaaacgaaacttctggaatattcccgaaagttggtcgggtgaggcgcaaaaggataaaaagccggtggaagcggagctgagcagatccgagccgggctggctgcaagcttgaattc

SEQ ID NO: 4 - DNA, 25 bp, synthetic construct
  source 1..25: mol_type = other DNA
cgaaacttctggaatattcccgaaa

(19) United States
(12) Patent Application Publication
(10) Pub. No.: US 2025/0101384 A1
(43) Pub. Date: Mar. 27, 2025

(54) TEMPERATURE-SENSITIVE INDUCTION OF THE MYOGENIC AND/OR ADIPOGENIC PROGRAMME IN CELLS FOR THE PRODUCTION OF MUSCLE AND/OR ADIPOSE TISSUE

(71) Applicant: UNIVERSITÀ DEGLI STUDI DI TRENTO, Trento (IT)

(72) Inventors: Stefano Augusto Maria BIRESSI, Trento (IT); Luciano CONTI, Galbiate (IT); Giulia FIORAVANTI, Trento (IT); Stefano LATTANZI

(21) Appl. No.: 18/730,244

(22) PCT Filed: Jan. 18, 2023
(86) PCT No.: PCT/IB2023/050422

§ 371 Date: Jul. 18, 2024

(30) Foreign Application Priority Data
Jan. 19, 2022 (IT) 102022000000788

(51) Int. Cl. C12N 5/077; A23L 13/00; C12N 15/86

(52) U.S. Cl. CPC C12N 5/0658; A23L 13/00; C12N 5/0653; C12N 15/86; C12N 2506/45; C12N 2510/00; C12N 2740/15043; C12N 2830/001

(57) ABSTRACT

The present invention relates to a method for the production of a muscle and/or adipose tissue, in particular cultured meat, providing the use of engineered cells capable of differentiating in muscle and/or adipose cells in a temperature-dependent manner. The invention also relates to expression vectors, engineered cells and kit for use in such production method.

### FIELD OF THE INVENTION

The present invention relates to a method for the production of a muscle and/or adipose tissue, in particular cultured meat, providing the use of engineered cells capable of differentiating in muscle and/or adipose cells in a temperature-dependant manner. The invention further relates to expression vectors, engineered cells and kit for use in such production method.

### STATE OF ART

In the last decade, the environmental impact of intensive farming, together with the ethical and economical involvements associated to the animals' sacrifice for food purposes, has pushed several researchers to explore alternative ways for producing products having features similar to meat. Ideally, such alternative approaches should lead to products (1) having nutritional and taste features similar to slaughtered meat, (2) obtainable on large scale, but (3) having low production costs and (4) limited environmental impact.

In the last few years, the production of cultured meat has attracted great interest by the society and scientific community. Most studies published in this field are centred on the expansion of myogenic cells (myoblasts) which are capable of forming in vitro muscle fibres, but which still have important limitations in terms of applicability in the production of cultured meat on wide scale.

As alternative to the use of myoblasts as cell source of the cultured meat, some studies have proposed the possible use of the stem cells, whose differentiation properties are not restricted to the formation of muscle fibers, such as for example induced pluripotent stem cells (iPSC), or somatic stem cells with myo/adipogenic capacity such as the mesenchymal cells. Such cells, with respect to myoblasts, have the advantage of having huge expansion capability and they are potentially capable of being grown/expanded in suspension. In contrast with the cultures of myoblasts, the pluripotent stem cells have to be suitably instructed to activate the differentiation process.

The cell engineering with constructs expressing crucial genes for implementing the myogenic programme is an approach which was proposed both in the field of regenerative medicine and in the field of the production of cultured meat. In some cases, such approach involves the use of regulatory sequences, or modified versions of the crucial genes, which are responsive to the administration of specific chemical molecules (for example, estradiol, doxycycline tectracycline, etc.). However, the latter, apart from having a relevant cost, have a strong toxicity risk for consumers and environment (Genovese et al. 2017).

For the just illustrated reasons, nowadays there is still a great need for detecting instruments which allow to optimize the production of a muscle and/or adipose tissue, in particular for the production of cultured meat so as to make it: (1) scalable at industrial level in economically sustainable manner, (2) capable of originating a product with nutritional features similar to slaughter meat, and (3) independent from the addition of chemical substances potentially toxic for consumers.

### SUMMARY OF THE INVENTION

The objective underlying the present invention is to provide expression vectors for engineering cells with myogenic and/or adipogenic capacity allowing to induce the differentiation thereof in muscle and/or adipose cells in effectively modulable manner from outside, so as to overcome the previously illustrated problems with reference to the state of art.

In particular, the authors of the present invention have developed expression vectors responsive to a physical parameter, the temperature change, capable of inducing the expression of myogenic and/or adipogenic master genes in cells in controlled manner. Such gene constructs offer the advantage of being able to optimize the production of muscle and/or adipose tissue, by avoiding the use in the cell culture medium of expensive and/or potentially toxic substances.

The vectors developed by the authors of the present invention are particularly suitable to modify genetically multipotent stem cells, for example induced pluripotent stem cells, as well as somatic cells with myo/adipogenic capacity, for example mesenchymal cells and they result to be able, in concert, to control the transition thereof from the expansion phase to the one differentiating in muscle or fat, alternatively, in a strictly temperature-dependent manner.

According to a preferred embodiment of the present invention, the vectors developed by the authors of the present invention comprise: a) a vector comprising (i) a promoter comprising 45 temperature-responsive elements, operably linked to (ii) a first nucleic acid sequence encoding for the regulatory factor of MyOD myogenesis and (iii) a second sequence of nucleic acid sequence encoding a chromatin remodelling agent (CRA) (in the present description also called as pTRE-45MyoD/CRA);
and b) a vector comprising (i) a promoter comprising 15 temperature-responsive elements, operably linked to (ii) a first nucleic acid sequence encoding for the regulatory factor of PPARγ adipogenesis and to (iii) a second nucleic acid sequence encoding for the regulatory factor of C/EBPβ adipogenesis (in the present description also called as pTRE-15PPARγ).

Advantageously, the inventors have found that the co-expression of the herein described pTRE-45MyoD/CRA and pTRE-15PPARγ vectors, together with the expression of a third vector comprising at least a nucleic acid sequence encoding for HSF1 under the control of the CAG constituting promoter (also called herein as pCAG-HSF1), allows to modulate the expression ratio between the genes of the myogenic programme and the adipogenic one in cells with myogenic and/or adipogenic capacity, and consequently it allows to regulate the proportion of these cells which differentiate in muscle or fat.

The use of the gene constructs developed by the authors of the invention then offers a big potential for the development of new methods for the production of muscle and/or adipose tissue, which can find application not only within the production of edible products intended for human consumption, such as cultured meat, but even in the field of the regenerative medicine. The methods, the present invention relate to, then allow not only to determine, by means of a temperature increase, a controlled transition from the proliferation step to the cell differentiation, but even to modulate the proportion of cells which differentiate in muscle or fat for the production of a muscle and/or adipose tissue.

In the context of the production of cultured meat, the method the present invention relates to, offers the opportunity to make such process safer, effective from the point of view of costs, industrially scalable, translatable to different animal species and capable of guaranteeing a control on the fat content of meat so as to optimize taste and nutritional properties thereof.

Therefore, the invention relates to:
an expression vector comprising (i) a temperature-responsive promoter operably linked to (ii) at least one nucleic acid sequence encoding for a regulatory factor of myogenesis and/or at least one nucleic acid sequence encoding for a regulatory factor of adipogenesis;
engineered cells having myogenic and/or adipogenic capacity, comprising an expression vector capable of inducing the differentiation of the above-mentioned cells in muscle and/or adipose cells in response to a temperature change;
an in vitro method for the preparation of a muscle and/or adipose tissue, which method comprises the following steps:
a) culturing the cells defined in any one of the herein described claims at a temperature suitable for the proliferation of said cells;
b) increasing the cultivation temperature so as to promote the differentiation of said cells in muscle and/or adipose cells;
c) culturing the differentiated cells so as to obtain said tissue;
a muscle and/or adipose tissue obtainable by means of a method according to any one of the herein described embodiments, preferably wherein said tissue is an edible product, in particular cultured meat;
a kit comprising at least one vector and/or cells according to any one of the herein described embodiments.

### BRIEF DESCRIPTION OF FIGURES

FIG. 1. Scheme of the production of cultured meat starting from undifferentiated cells in a manner controlled by physical parameters. Schematic representation of a method according to an embodiment of the present invention, to control the transition from the expansion phase of pluripotent stem cells and/or somatic cells with myo/adipogenic capacity to that of differentiating in muscle or alternatively in fat. Such transition depends upon the operation of 2 vectors according to an embodiment described herein, illustrated in FIGS. 2 and 3. Such vectors guarantee that the temperature increase determines the expression of promoter genes of the myogenic (for example MyoD) and adipogenic (for example PPARγ) differentiation, together with the expression of genes which modify the accessibility to DNA and favour the effectiveness thereof.

FIG. 2. Vector expressing muscle differentiation factors in a temperature-dependent manner according to an embodiment. Schematic diagram of a bicistronic lentiviral vector devised to induce the expression of a master gene of the myogenic regulation (for example MyoD) and of a gene encoding a chromatin remodelling agent (CRA). The transcription of the two genes is under control of responsive-to-temperature regulatory sequences (TRE) derived from the HSP70 (heat shock protein 70) promoter. The TRE sequences are multimerized 45 times to allow an intense expression in a low temperature interval. In order to allow the selection respectively in bacteria and eukaryotic cells, cassettes were inserted expressing genes encoding the resistors such as for example kanamycin (KANr) and blasticidin (Blar). The vector includes an origin of bacterial replication and the sequences required to pack in the viral particles.

FIG. 3. Vector expressing adipose differentiation factors in a temperature-dependent manner according to an embodiment. Schematic diagram of the lentiviral vector designed to induce co-expression of the PPARγ (peroxisome proliferator-activated receptor gamma) and C/EBPβ adipogenic master genes. The transcription of the two genes is under control of the responsive-to-temperature regulatory sequences (TRE) derived from the HSP707 (heat shock protein 70) promoter. The TRE sequences are multimerized 15 times to allow an expression of medium intensity in a low temperature range. In order to allow the selection in bacteria and eukaryotic cells, respectively, cassettes were inserted expressing genes encoding resistors such as for example kanamycin (KANr) and blasticidin (Blar). The vector includes an origin of bacterial replication and the sequences required to pack in the viral particles.

FIG. 4. Vector expressing heat shock factor 1 (HSF1). Schematic diagram of the lentiviral vector designed to induce the expression of the heat shock factor 1 (HSF1) gene. The HSF1 transcription is under control of constitutive regulatory sequences such as CAG sequences (CMV early enhancer fused to modified chicken β-actin promoter). In order to allow the selection in bacteria and eukaryotic cells, respectively, cassettes were inserted expressing genes encoding the resistors such as for example kanamycin (KANr) and blasticidin (Blar). The vector includes an origin of bacterial replication and the sequences required to pack in the viral particles. Used in combination with the vectors described in FIGS. 2 and 3, this vector amplifies the transcriptional capability thereof.

FIG. 5. Scheme of how temperature modules expansion and differentiation of cells in the process for producing cultured meat according to an embodiment. Scheme of a possible process for producing cultured meat using the constructs described in this invention. The plant includes a bioreactor where the pluripotent stem cells or the somatic cells with genetically modified myo/adipogenic capacity are expanded and a bioreactor for the differentiation containing the “scaffolds” to give structure to the product. In the expansion bioreactor the temperature is 37° C. A continuous stirring guarantees oxygenation of the whole plant and the homogeneous distribution of base nutrients (that is, carbohydrates, aminoacids, vitamins, etc.). A transient temperature increase of the medium at the inlet in the differentiation bioreactor can be obtained with a system of resistors and determines the induction of genes responsible for differentiation. The different reactivity to temperature of the constructs expressing the genes of the myogenic or adipogenic differentiation allows to modulate the proportion of cells which differentiate alternatively in muscle or in fat, thus by controlling the features of the final product.

FIG. 6. Sequence of 154 bps which comprises upstream 15 HSE sites (nGAAn) and the minimum promoter of HSPA6 (72 pbs, grey).

FIG. 7. Sequence of 204 bps which comprises upstream 45 HSE sites (nGAAn) and the minimum promoter of HSPA6 (72 pbs, grey).

FIG. 8. Lentiviral vector. The GFP Turbo was inserted downstream of the promoter 15HSE-Hsp70 as promoter activating marker. The mCherry fluorescent protein is used as plasmid transfection marker.

FIG. 9. Lentiviral vector. The GFP Turbo was inserted downstream of the promoter 45HSE-Hsp70 as promoter activating marker. The mCherry fluorescent protein is used as plasmid transfection marker.

FIG. 10. Lentiviral plasmid. The HSF1 gene was inserted downstream of the CAG promoter. The TagBFP2 fluorescent protein is used as plasmid transfection marker.

FIG. 11. Increase in percentage of cells with active promoter (GFP+ve) from 37° C. to 43° C. in cells (A) with HSF1 plasmid and in cells (B) transfected with HSF1 plasmid.

FIG. 12. Increase in intensity in cells with active promoter (GFP+ve) from 37° C. to 43° C. in normalized data. The data were normalized on the GFP and mCherry (GFP median/mCherry median) intensities and the increase was normalized in cells (A) without HSF1 plasmid and in cells (B) transfected with HSF1 plasmid.

FIG. 13. Histogram representing the GFP intensity (promoter activating indicator) of several cell populations at 37° C. The graph shows how the increase in number of HSE repetitions and the HSF1 addition decreases the promoter basal activation.

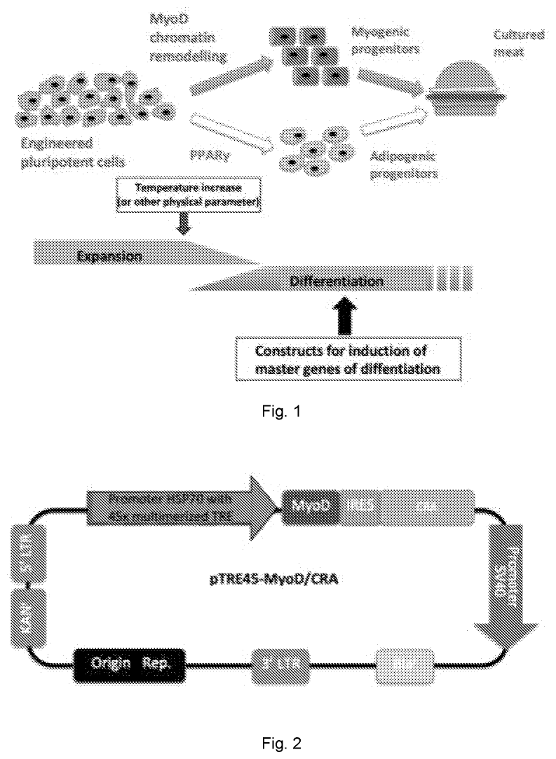

### GLOSSARY

The terms used in the present description are as generally understood by the person skilled in the art, unless otherwise indicated.

The terms “promoter” or “promoter sequence” are used in the present application interchangeably and they relate to a DNA sequence capable of controlling the expression of a nucleotide sequence operably linked to said promoter/sequence of a promoter.

In particular, the term “temperature-responsive promoter” as used in the context of the present invention, refers to a promoter or a promoter sequence having activity which can be induced or controlled externally by means of temperature change. In other terms, a “temperature-responsive promoter” is a promoter which has not the capability of controlling the expression of the nucleotide sequence operably linked thereto before a determined temperature value or range of values is reached and which, on the contrary, has the capability of controlling the expression of the above-mentioned sequence once this temperature or range of values has been reached.

The expression “identity %” as used in the context of the present description with respect to a reference nucleotide sequence is defined as the percentage of nucleotides in a candidate sequence which are identical with respect to the nucleotides in the reference nucleotide sequence, after alignment of the sequences and introduction of spaces—if required—so as to reach the maximum sequence identity percentage.

The expression “myogenesis regulatory factor”, in the context of the present invention, comprises a protein, agent or transcription factor capable of regulating or inducing the myogenesis factor, that it the process of formation of muscle tissue. Such definition, for example, comprises nuclear phosphoproteins with function of transcription transactivator for specific genes of the muscle cell, such as myosin, muscle creatine kinase (MCK) and the nicotinic acetylcholine receptor (AChR). The myogenesis regulatory factors belong to the family of transcription factors called “b-HLH proteins” (Basics helix-loop-helix): all proteins of this family are capable of binding to DNA in similar sites, by activating specific genes of the muscle.

The expression “adipogenesis regulatory factor”, in the context of the present description”, comprises a protein, agent or transcription factor which is involved in the regulation or induction of adipogenesis process, that is the process therethrough the formation of adipose tissue takes place, in particular through the differentiation of the preadipocytes in adipocytes.

The term “plasmid”, as used herein, relates to a DNA circular filament supercoiled in double helix, capable of transporting a nucleic acid of interest inside a target organism. Some plasmids are capable of autonomous replication inside one target cell, inside thereof they were introduced. Other vectors can be integrated in the genome of a host cell and, then, can be replicated with the host genome. In particular, some plasmids are capable of controlling the expression of genes thereto they result to be operably linked.

The term “tissue”, as explained therein, has the meaning commonly used in biology, that is it defines a set of structurally similar cells, associated by function. The tissue constitutes a higher level of cell organization: several different tissues can associate therebetween to form further organized structures. The muscle tissue by definition consists of muscle cells whereas the adipose tissue is formed by cells called adipocytes.

### DETAILED DESCRIPTION OF THE INVENTION

As previously mentioned, the authors of the present invention have developed temperature-responsive gene constructs for engineering cells with myogenic and/or adipogenic capacity, with the purpose of inducing the differentiation of these cells in muscle and/or adipose cells in an effectively controlled manner.

### Vectors

In a first aspect, therefore, the present invention provides an expression vector comprising (i) a temperature-responsive promoter operably linked to (ii) at least one nucleic acid sequence encoding for a regulatory factor of myogenesis and/or at least one nucleic acid sequence encoding a regulatory factor of adipogenesis.

A “temperature-responsive promoter”, as described herein, preferably is a promoter having the capability of inducing the expression of at least a nucleic acid sequence encoding for a regulatory factor of myogenesis and/or at least a nucleic acid sequence encoding a regulatory factor of adipogenesis operably linked thereto.

A “temperature-responsive promoter” suitable to be used in an expression vector according to the present invention is a promoter derived from a heat-inducible gene, preferably it is a heat-shock promoter. In a preferred embodiment, the promoter derived from HSP70 is used, wholly described in the scientific article of Brade et al. (2000, “Heat-directed gene targeting of adenoviral vectors to tumour cells”).

According to a preferred aspect of the present invention, said temperature-responsive promoter comprises a plurality of temperature-responsive elements (heat shock elements, HSE, alternatively called herein even under the acronym TRE). Temperature-responsive elements are consensus sequences present in the promoters of inducible heat-shock proteins (HSPs).

According an aspect of the invention, said plurality of HSE comprises a plurality of 5′-NGAAN-3′ nucleic acid sequences, wherein the letter N represents any nucleotide.

Preferably, a HSE sequence consists of at least three pentameric units of the 5′-NGAAN-3′ sequence. Still in a preferred embodiment, the temperature-responsive promoter comprises 9 repetitions of the sequence containing 5 HSE: CGAAA CTTCT GGAAT ATTCC CGAAA (SEQ ID No 4).

A vector according to any one of the herein described embodiments, preferably comprises a temperature-responsive promoter wherein said HSE are in number at least equal to 2 or higher. In a preferred embodiment of the invention, said plurality of HSE comprises a number equal to 15 of HSE, or a number equal to 45 of HSE.

Still according to an aspect of the present invention, the temperature-responsive promoter according to any one of the herein described variants can further comprise a nucleic acid sequence of HSPA6 promoter, operably linked to said plurality of HSE.

The HSPA acronym derives from English “Heat shock protein family A (Hsp70) Member 6”.

According to an aspect of the present invention, said temperature-responsive promoter comprises or consists of a nucleotide sequence having SEQ ID NO. 1.

According to an aspect of the present invention, said temperature-responsive promoter comprises or consists of a nucleotide sequence having more than 90%, preferably more than 95%, preferably more than 99% of identity with sequence SEQ ID No 1.

Preferably, the temperature-responsive promoter usable in a vector according to any one of the herein described embodiments has the nucleotide sequence SEQ ID No. 2 or SEQ ID No. 3.

It is to be meant that the sequence of the temperature-responsive promoter according to any one of the herein described embodiments could include one or more mutations, provided that these do not compromise the activity and/or functionality thereof as herein described.

According to an aspect of the invention, the temperature-responsive promoter is a synthetic promoter.

Any nucleotide sequence encoding for a regulatory factor of myogenesis and/or adipogenesis can be inserted in the vector of the invention.

It was widely demonstrated that the muscle differentiation can be induced by inducing master genes of the myogenic programme, such as MyoD, myogenin, MYF-5 and MRF4, PAX3 and PAX7.

Therefore, according to an aspect of the present invention, said regulatory factor of myogenesis is selected among MyoD, myogenin, MYF-5, MRF4, PAX3 and PAX7, in particular it is one or more of MyOD, myogenin, MYF-5, MRF4, PAX3 and PAX7. The aminoacidic sequence of each one of said exemplified factors is known in the art, and therefore a nucleic acid encoding for each one of said sequences can be easily designed.

MyoD, also known as “myoblast determination protein 1” is a nuclear protein belonging to the family of the regulatory factors of myogenesis, which regulates the differentiation of the muscle cells by inducing the cell cycle arrest, a prerequisite to start the myogenesis process. This protein is also involved in the muscle regeneration.

Preferably, the nucleic acid sequence encoding for a regulatory factor of myogenesis used in a vector according to any one of the herein described embodiments is a sequence of cisgenic nucleic acid encoding for MyoD, that is a sequence deriving from a donor animal organism of the same species of the cells which will be modified with said vector according to any one of the herein described embodiments. In other terms, said encoding sequence preferably is a nucleic acid sequence wholly belonging to the species of the cells receiving the above-mentioned vector or to one similar thereto, according to any one of the herein described embodiments.

By pure way of example, said sequence is the sequence identifiable in Gene Bank by means of Gene ID: 281938.

According to an aspect of the present invention, said regulatory factor of adipogenesis is selected from PPARγ and C/EBPβ.

The gamma receptor activated by the proliferators of peroxisomes (PPARγ or PPARG, from English “peroxisome proliferator-activated receptor gamma”) also known as glitazone receptor or NR1C3, is a nuclear receptor of second type which in the human species is encoded by the gene PPARG. The PPARγ factor was identified as an important regulator of adipogenesis, in fact the protein encoded by the gene PPARγ regulates the differentiation of adipocytes. This factor in particular provides a dynamic and specific regulation during the differentiation from a precursor cell to wholly differentiated adipocyte.

The C/EBPβ protein, also known as “CCAAT/enhancer binding protein-β”, is a transcription factor which is commonly associated to the regulation of cell proliferation, encoded in man by CEBPB gene.

Preferably, the nucleic acid sequence encoding for a regulatory factor of adipogenesis used in a vector according to any one of the herein described embodiments comprises a first sequence encoding for the regulatory factor of adipogenesis PPARγ and a second nucleic acid sequence encoding for the regulatory factor of adipogenesis C/EBPβ.

Still more preferably, the nucleic acid sequence encoding for a regulatory factor of adipogenesis used in a vector according to any one of the herein described embodiments comprises a first cisgenic nucleic acid sequence encoding for the regulatory factor of adipogenesis PPARγ and a second cisgenic nucleic acid sequence encoding for the regulatory factor of adipogenesis C/EBPβ, that is sequences deriving from a donor animal organism of the same species of the cells which will be modified with said vector according to any one of the herein described embodiments.

By pure way of example, the nucleic acid sequence encoding for the regulatory factor of adipogenesis PPARγ is the sequence identifiable in Gene Bank by means of Gene ID: 281993.

A vector according to any one of the herein described embodiments can further comprise a nucleic acid sequence encoding a chromatin remodelling agent (CRA). A preferred example of CRA agent is represented by the BAF60C (BRGI/Brm-associated factor of 60 kDa, subunit C) factor.

Preferably, the nucleic acid sequence encoding for a CRA agent usable in a vector according to any one of the herein described embodiments is the sequence encoding for a SWI/SNF (from English Swich/Sucrose Non-fermentable) agent, a sub-family of complexes capable of remodelling ATP-dependent chromatin which are present in the eukaryotes, for example the SMARCD3 gene. By pure way of example, the nucleic acid sequence encoding for a CRA agent usable in a vector according to any one of the herein described embodiments is the sequence identifiable in Gene Bank by Gene ID: 777769.

According to a preferred aspect of the invention, said nucleic acid sequence encoding a CRA agent is operably linked to at least a nucleic acid sequence encoding for a regulatory factor of myogenesis, in particular MyoD; in this way, the expression of CRA agent allows to favour the action of the regulatory factor of myogenesis and then the myogenic conversion process.

Preferably, said nucleic acid sequence encoding a CRA agent is operably linked to said at least a nucleic acid sequence encoding for a regulatory factor of myogenesis, in particular MyoD, by means of a IRES nucleic acid sequence. In other terms, the insertion of the sequence encoding a CRA, such as for example BAF60C, preferably is performed downstream of the sequence(s) encoding MyoD, with a IRES sequence acting as a bridge to guarantee the in-tandem expression.

According to a preferred embodiment of the invention, said vector is selected among:
a) a vector comprising (i) a promoter comprising 45 temperature-responsive elements, operably linked to (ii) a first nucleic acid sequence encoding for the regulatory factor of myogenesis MyoD and to (iii) a second nucleic acid sequence encoding a chromatin remodelling agent (CRA); wherein said second nucleic acid sequence is operably linked to said first nucleic acid sequence by means of an IRES sequence; preferably wherein said promoter has the nucleotide sequence SEQ ID No. 3; and b) a vector comprising (i) a promoter comprising 15 temperature-responsive elements, operably linked to (ii) a first nucleic acid sequence encoding for the regulatory factor of adipogenesis PPARγ and to (iii) a second nucleic acid sequence encoding for the regulatory factor of adipogenesis C/EBPβ; preferably wherein said promoter has the nucleotide sequence SEQ ID No. 2.

It is preferable that the nucleic acid sequences encoding for MyoD, PPARγ and C/EBPβ present in the vectors according to the above-mentioned preferred embodiment are cisgenic nucleic acid sequences, that is wholly belonging to the species of the cells receiving the above-mentioned vectors or to one similar thereto.

FIG. 2 illustrates schematically an embodiment of the vector a) as described in the present application, also called pTRE-45MyoD/CRA; this vector includes cDNA expressing MyoD under the control of a promoter derived from HSP70 gene characterized by the repetitions of 45 temperature-responsive elements and robustly inducible by temperature increase of few degrees Celsius. The vector pTRE-45MyoD/CRA includes a high number (45) of HSE (or TRE) repetitions. In the specific, the promoter inducible by the temperature present in this vector includes 9 repetitions of the sequence containing 5 HSE (or TRE): CGAAA CTTCT GGAAT ATTCC CGAAA (SEQ ID No 4). The vector pTRE-45MyoD/CRA, apart from MyoD, results to be able to express even a chromatin remodelling agent and then capable of favouring the MyoD action and then the myogenic conversion in a temperature increase-depending manner.

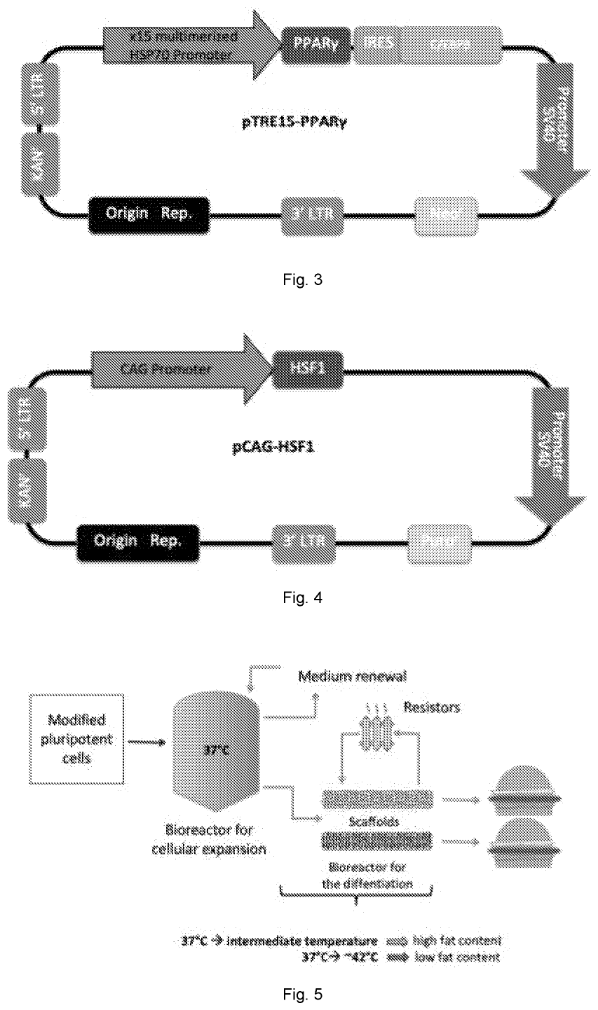

FIG. 3 illustrates schematically an embodiment of the vector b) as described in the present application, also called pTRE15-PPARγ; such vector expresses the master genes of the PPARγ and C/EBPβ adipogenic programme. In this vector the above-mentioned genes of the adipogenic programme are under the control of a promoter containing 15 HSE (or TRE) given by repeating 3 times the sequence containing 5 HSE (or TRE): CGAAA CTTCT GGAAT ATTCC CGAAA (SEQ ID No 4). The authors have found that the vector pTRE-45MyoD/CRA has a greater effectiveness in response to temperature than vector pTRE15-PPARγ.

An alternative embodiment according to the present invention relates to a vector comprising (i) a promoter comprising 45 temperature-responsive elements, operably linked to (ii) a first nucleic acid sequence encoding for the regulatory factor of adipogenesis PPARγ and to (iii) a second nucleic acid sequence encoding for the regulatory factor of adipogenesis C/EBPβ; preferably wherein said promoter has the nucleotide sequence SEQ ID No. 2.

A vector according to any one of the herein described embodiments is a system capable of transporting nucleic acid sequences of interest inside a foreign organism and of allowing the expression of genes or integration of the same inside the target organism. Preferably, said organism is a cisgenic organism, that is an organism which has been modified by means of one or more vectors according to any one of the herein described embodiments, which vectors comprise gene or nucleic acid sequences coming from organisms of the same species or however similar.

Once inside a target cell, the vector according to any one of the herein described embodiments can be useful for example to keep DNA heterologous inside the cell or, alternatively, it can act as unit for the DNA replication.

According to an aspect of the invention, said expression vector can be of viral or non-viral type, for example a plasmid.

According to an additional aspect of the present invention, a vector according to any one of the herein described embodiments is a viral vector, in particular it is a viral vector based on a lentivirus, an adenovirus, a retrovirus or a herpes simplex virus. In a preferred embodiment according to the present invention, said vector is a lentiviral vector.

The lentiviral vector can be in the form of a DNA recombinant molecule, for example a plasmid. The lentiviral vector alternatively can be in the form of a vector of lentiviral particle, comprising for example one or more molecules of RNA in a complex of lentiviral proteins or other proteins. The lentiviral vectors derive from lentivirus, in particular from virus of human immunodeficiency (HIV-1 or HIV-2), monkey immunodeficiency virus (SIV), equine infective encephalitis virus (EIAV), goat viral encephalitis arthritis virus (CAEV), bovine immunodeficiency virus (BIV) and feline immunodeficiency virus (FN), which are modified to remove genetic determinants involved in the pathogenicity and to introduce new determinants useful to obtain determined effects of interest.

A vector according to any one of the herein described embodiments can be produced through any one of the techniques known to a person skilled in the art in the field of the genetic engineering and biotechnologies.

### Cells

The present invention also relates to engineered cells having myogenic and/or adipogenic capacity comprising an expression vector capable of inducing the differentiation of the above-mentioned cells in muscle and/or adipose cells in response to a temperature change.

Under the expression “engineered cells having myogenic and/or adipogenic capacity”, in the context of the present invention, one refers to isolated cells which are capable of differentiating in muscle and/or adipose cells, that is isolated cells having the potential of developing in muscle and/or adipose cells.

An expression vector which is capable of inducing the differentiation of the above-mentioned cells in muscle and/or adipose cells in response to a temperature change is any vector having the capability of activating the differentiation of cells including it in muscle and/or adipose cells upon reaching a determined activation temperature value.

In particular, a suitable expression vector is a vector comprising a temperature-responsive promoter, for example a promoter according to any one of the embodiments previously illustrated in the present description.

The engineered cells according to the present invention can include an expression vector according to any one of the herein described embodiments. Preferably, the above-mentioned cells are infected or transduced, transformed or transfected with an expression vector according to any one of the herein described embodiments.

The term “transformed or transfected” relates to host cells which were subjected to a transformation or transfection process. The transformation or transfection is the process by which exogen genetic material is introduced in host cells. Such process can be performed in vitro or in vivo. In particular, the cells, the invention relates to, can be infected, transformed and/or transfected by using any one of the techniques known to a person skilled in the art, for example a chemical or physical method. The cells can even be examined so as to select the cells which really transport the vector or genes of interest, by using conventional cell screening techniques, such as Southern blots and/or PCR, or by using specific markers.

Infected, transformed or transfected cells correctly result to be capable of expressing the genes or other polynucleotides and/or parts of the vector according to the invention inserted therein.

According to an aspect of the present invention, a vector according to any one of the herein described embodiments is integrated in the chromosome of the above-mentioned engineered cells comprising it.

The engineered cells according to the invention can be obtained by starting from an animal, preferably a non-human mammal. According to an aspect of the present invention, said cells are non-human cells. The herein described cells with myogenic and/or adipogenic capacity preferably are cells of any edible animal species, for example livestock or poultry. Such cells can be for example cells obtained from species of pig, sheep, cattle, fish, shellfish, bird or similar.

Still more preferably, the cells having myogenic and/or adipogenic capacity according to the present invention comprise one or more vectors according to any one of the herein described embodiments, which vectors comprise genes or nucleic acid sequences coming from organisms of the same species of the above-mentioned cells or however similar.

Engineered cells with myogenic and/or adipogenic capacity suitable to be infected, transduced, transformed or transfected with a vector according to any one of the herein described embodiments are pluripotent stem cells, multipotent stem cells and/or mesenchymal stem cells. Such cells, with respect to the myoblasts conventionally used in the art, have the advantage of having huge expansion capability. Moreover, such cells can be grown and/or expanded in suspension.

The pluripotent stem cells are primitive, not specialized cells, provided with the capability of originating one or more lines or cell types through the cell differentiation process. The mesenchymal stem cells (MSC) are an example of tissue or “adult” stem cells. They are multipotent cells, that is capable of producing different types of specialized cells of the body, but not all types.

Still according to an aspect of the present invention, the engineered stem cells are pericytes, for example porcine pericytes. The pericyte is a type of undifferentiated mesenchymal cell with contractile function, which partially surrounds the sub-endothelial cells of the capillaries and of the venules.

Engineered stem cells according to the present invention can be even induced pluripotent stem cells: these are a type of pluripotent stem cell derived from a non-pluripotent cell, typically an adult somatic cell, through the manipulation of some genes.

In an additional embodiment, cells with myogenic and/or adipogenic capacity according to the present invention comprise fibroblasts, hepatocytes and/or any other type of cell known to a person skilled in the art having myogenic and/or adipogenic capacity.

The temperature increase controls the gene expression mostly at transcriptional levels, through the interaction of the HSF1 (“heat shock factor 1”) factor, activated by the HSE (or TRE) sequences such as those described in the present application, present in the temperature-inducible gene promoters. HSF1 is expressed endogenously in most cell types. However, the cell modification with a vector which is capable of expressing HSF1 allows to increase the difference in responsiveness to temperature of vectors according to any one of the herein described embodiments.

Therefore, the cells according to any one of the herein described embodiments can further include a vector comprising at least a nucleic acid sequence encoding for HSF1. Preferably, the nucleic acid sequence encoding for HSF1 is the sequence encoding for HSF1 deriving from a donor animal organism of the same species of cells which will be modified with said vector according to any one of the herein described embodiments. By pure way of example, said sequence is the sequence identifiable in Gene Bank by means of Gene ID: 506235.

According to a preferred aspect, the nucleic acid sequence encoding for HSF according to any one of the herein described embodiments is under control of a constitutive promoter. A constitutive promoter can be any promoter suitable to induce the expression of HS1 factor, preferably it is the CAG promoter. FIG. 4 illustrates schematically an embodiment of a vector capable of expressing HSF1, called pCAG-HSF1.

A preferred aspect according to the present invention in particular relates to engineered cells according to any one of the herein described embodiments, comprising the following vectors:
a) a vector comprising (i) a promoter comprising 45 temperature-responsive elements, operably linked to (ii) a first nucleic acid sequence encoding the regulatory factor of myogenesis MyoD and to (iii) a second nucleic acid sequence encoding a chromatin remodelling agent (CRA); wherein said second nucleic acid sequence is operably linked to said first nucleic acid sequence by means of an IRES sequence; preferably wherein said promoter has the nucleotide sequence SEQ ID No. 3;
b) a vector comprising (i) a promoter comprising 15 temperature-responsive elements, operably linked to (ii) a first nucleic acid sequence encoding for the regulatory factor of adipogenesis PPARγ and to (iii) a second nucleic acid sequence encoding for the regulatory factor of adipogenesis C/EBPβ; preferably wherein said promoter has the nucleotide sequence SEQ ID No. 2; and
c) a vector comprising at least one nucleic acid sequence encoding for HSF1 under control of the constitutive promoter CAG according to any one of the herein described embodiments.

The co-expression of vector c) with the vectors a) and b) according to the present invention (an embodiment thereof is illustrated respectively in FIGS. 4, 2 and 3) allows to amplify the capability of these two vectors of inducing the myogenic or adipogenic programme, respectively, in cells in a temperature-dependant manner.

### Methods

An additional aspect of the invention relates to an in vitro method for the preparation of a muscle and/or adipose tissue, which method comprises the following step:
a) culturing the cells as defined in any one of the herein described embodiments at a temperature suitable for the proliferation of said cells;
b) increasing the cultivation temperature so as to promote the differentiation of said cells in muscle and/or adipose cells;
c) culturing the differentiated cells so as to obtain said tissue.

In the method, the present invention relates to, by increasing transiently the temperature in the culture medium in step b) with respect to step a), and optionally by varying the temperature during the differentiation process within a pre-established range, it will be possible to modulate the gene expression ratio of the myogenic and/or adipogenic programme in cells, and consequently to modify in a controlled manner the proportion of cells which differentiate in muscle or fat.

In fact, the different reactivity to temperature of the vectors expressing the genes of the myogenic or adipogenic differentiation allows to modulate the proportion of cells which alternatively differentiate in muscle or fat, thus by controlling the features of the final product.

The steps a) and c) of the herein described method preferably are carried out in separate reactors or bioreactors. Under the terms “reactors” or “bioreactors”, in the context of the present invention, devices or systems are meant allowing the cultivation of the above-mentioned cells on laboratory scale, on medium or large scale. Examples of reactors which can be used in a method according to the present invention include reactors having a capacity from 1 L to 10,000 L.

According to an aspect of the invention, the step a) is carried out in at least a first reactor or bioreactor comprising a culture medium. The person skilled in the art, based upon the type of cell used in the herein described method, could be able to select the culture medium most suitable to guarantee the cell proliferation, among those known in the art.

According to an additional aspect of the invention, the step a) can be carried out in two or more distinct reactors or bioreactors, in particular in at least (a′) a first reactor or bioreactor used for culturing cells having myogenic capacity according to any one of the herein described embodiments and in at least (a″) a second reactor or bioreactor used to culturing cells having adipogenic capacity according to any one of the herein described embodiments.

The step b) of the method according to any one of the herein described embodiments can be carried out inside the same reactor (or same reactors) used in step a) or in one or more different reactors or containers in which the cells cultivated in step a) have been transferred.

Preferably, the cultivation step a) and the cell differentiation step c) of a method according to any one of the herein described embodiments are carried out in reactors separated therebetween.

Step c) of a method according to any one of the herein described embodiments preferably is carried out in at least a second reactor comprising a culture medium, which second reactor is operably linked to means for the temperature regulation in said second rector.

According to an aspect of the invention, step c) of a method according to any one of the herein described embodiments is carried out in two or more distinct reactors or bioreactors, in particular in at least (c′) a first reactor or bioreactor used for differentiating cells having myogenic capacity according to any one of the herein described embodiments, previously cultivated in step (a′), and in at least (c″) a second reactor or bioreactor used to differentiate cells having adipogenic capacity according to any one of the herein described embodiments, previously cultivated in step (a″).

Preferably, the plant reactors, containers or components used in steps a), b) and c) according to any one of the herein described embodiments are all operably linked to means for regulating the temperature in the above-mentioned reactors.

Suitable means for regulating the temperature can include any system or device allowing to fix the temperature of the plant reactor, container or component considered at a pre-established value and/or to vary the temperature in such plant reactor, container or component in a controlled manner during the cell differentiation process. Preferably, means for the temperature regulation comprises a plurality of resistors.

According to an aspect of the invention, the reactor (or more reactors) used in step a) is (are) operably linked to the reactor (or more reactors) used in step c) so as to allow the cell transfer, at the end of the proliferation phase, inside the reactor (or more reactors) used for the cell differentiation.

Step b) of the herein described method preferably is carried out at a temperature comprised between 2° and 45° C.

According to a preferred embodiment, the temperature can be suitably modified during the differentiation process, for example during step b) or c) for example it can be increased gradually during the differentiation process so as to modulate the cell proportion differentiating in muscle or adipose cells.

With the purpose of conferring a pre-defined three-dimensional shape to the muscle and/or adipose tissue, in the method according to any one of the herein described embodiments, step c), that is the cell differentiation phase, can be carried out inside one or more scaffolds or three-dimensional moulds which are capable of conferring a predetermined shape to said tissue.

Steps a), b) and c) of the method preferably are carried out in a liquid culture medium.

According to an aspect of the invention, the herein described method can include an additional step: d) preparing an edible product with the tissue obtained in said step c).

The method according to any one of the herein described embodiments can further include a step, preceding the cultivation step a), in which the engineered cells according to any one of the previously described embodiments are infected, transduced, transformed or transfected with one or more vectors according to the present invention.

According to a preferred aspect of the invention, the muscle and/or adipose tissue obtainable by means of a method according to any one of the herein described embodiments is cultured meat or synthetic meat.

The present invention also relates to a muscle and/or adipose tissue obtainable in vitro by a method according to any one of the herein described embodiments.

Preferably said tissue is an edible product, in particular suitable for the human or animal consumption, preferably it is cultured or synthetic meat. By pure way of example, said edible product can be in form of animal feed.

According to an additional aspect of the invention, said tissue is a tissue suitable to be used in the field of the regenerative medicine, for example in a method for repairing or replacing a damaged tissue in a patient requiring it.

The invention further relates to a kit comprising at least a vector and/or cells according to any one of the herein described embodiments.

The invention kit could comprise even reagents and/or means for culturing the cells according to any one of the herein described embodiments, optionally it will comprise even use instructions.

According to a preferred embodiment, said kit will comprise at least the following vectors:
a) a vector comprising (i) a promoter comprising 45 temperature-responsive elements, operably linked to (ii) a first nucleic acid sequence encoding the regulatory factor of myogenesis MyoD and to (iii) a second nucleic acid sequence encoding a chromatin remodelling agent (CRA); wherein said second nucleic acid sequence is operably linked to said first nucleic acid sequence by means of an IRES sequence; preferably wherein said promoter has the nucleotide sequence SEQ ID No. 3;
b) a vector comprising (i) a promoter comprising 15 temperature-responsive elements, operably linked to (ii) a first nucleic acid sequence encoding the regulatory factor of adipogenesis PPARγ and to (iii) a second nucleic acid sequence encoding the regulatory factor of adipogenesis C/EBPβ; preferably wherein said promoter has the nucleotide sequence SEQ ID No. 2; and
c) a vector comprising at least one nucleic acid sequence encoding for HSF1 under control of the constitutive promoter CAG according to any one of the herein described embodiments.

It is preferable that the nucleic acid sequences encoding for MyoD, PPARγ, C/EBPβ and HSF1 present in the vectors according to the above-mentioned preferred embodiment are cisgenic nucleic acid sequences, that is wholly belonging to the species of the cells receiving the above-mentioned vectors or to one similar thereto.

The present invention also relates to the in vitro use of a vector, of cells and/or of a kit according to any one of the herein described embodiments for the production of a muscle and/or adipose tissue, preferably wherein said tissue is an edible product, in particular cultured meat.

### EXAMPLES

Some not-limiting examples of the method according to the present invention are shown herebelow by way of illustration.

### Example 1

### Methods

### Production of Plasmids

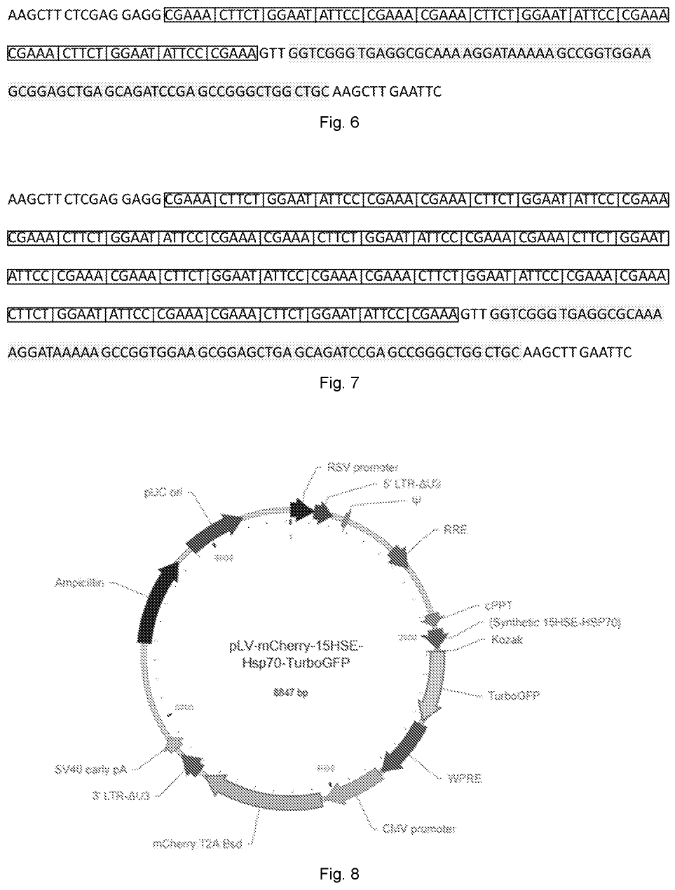

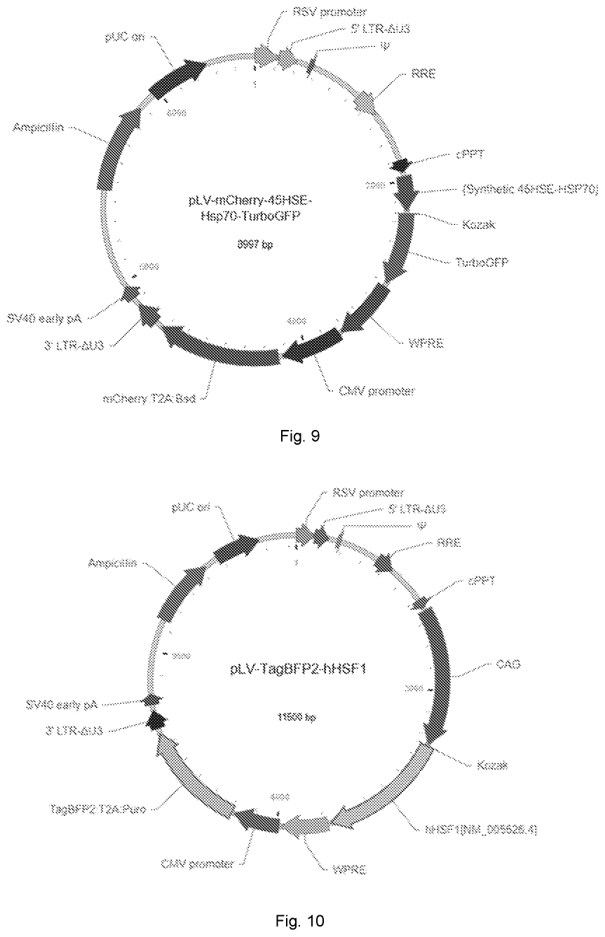

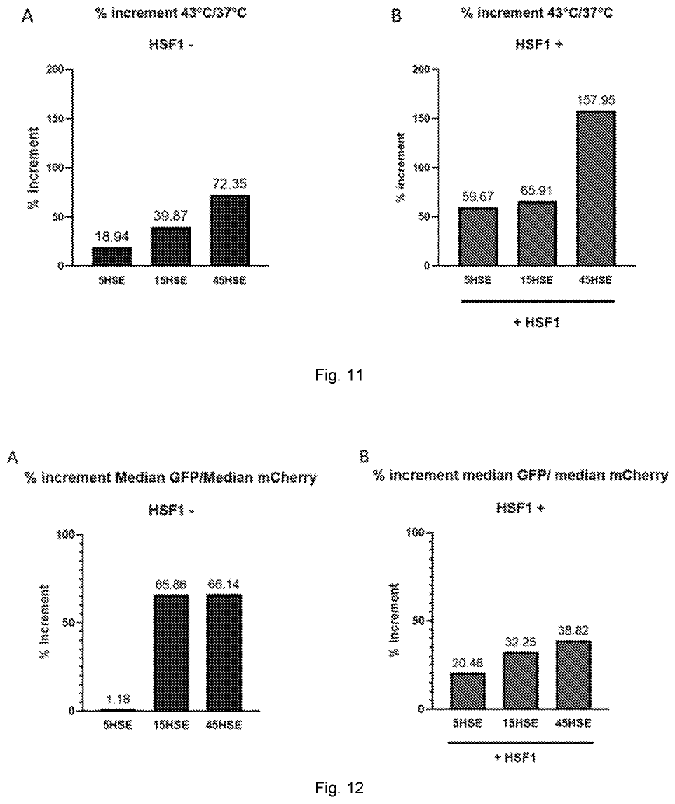

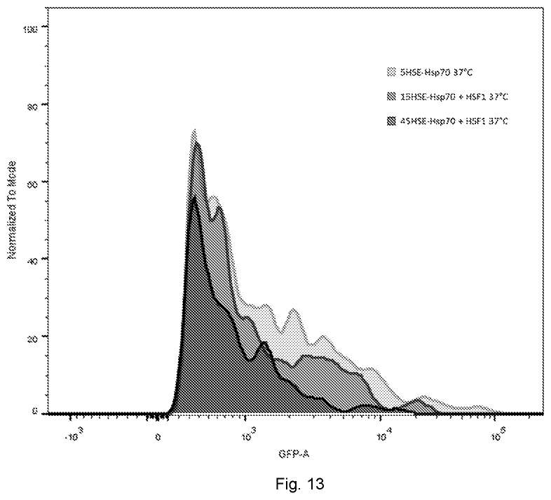

The plasmids were synthetized by VectorBuilder. The sequences of promoters were devised based upon the minimum promoter of HSPA6 (72 (bps), by adding upstream a repetition of 15 or 45 heat shock elements (HSE) (nGAAn) (FIG. 6-9).

A plasmid was also synthetized containing the gene encoding for the heat shock factor 1 (HSF1) (FIG. 10). HSF1 is a transcription factor which is usually in an inactive state and activates when the cells are exposed to heat. In active form, HSF1 binds to the HSE sequences on the Hsp70 promoter and induces the transcription thereof.

Culture of Porcine Pericytes The porcine pericytes were given by C. Gargioli (Università di Roma Tor Vergata, Rome, Italy). The cells were cultivated in MEM α with nucleosides and GlutaMAX™ (32571028, Gibco), 100 U/mL Penicillin, 100 g/mL Streptomycin (15140-122, Gibco), 20% Foetal Bovine Serum (FBS) (26140079, Gibco). The cells were detached every 3 days by using 0.25% trypsin-EDTA (25200056, Gibco).

### Nucleofection

The cells were plated two days before transfection (day −2). On day 0, the cells were detached with trypsin and centrifuged at 200 g×5 minutes. The pellet was resuspended in MEM α+20% FBS, and a count of the same cells was performed.

Subsequently, 2×10⁵ cells were diluted in 100 μL of Nucleofection Magic Buffer (KCl 5 mM, MgCl₂ 15 mM, Glucose 10 mM, K₂HPO₄ PH 7.2 120 mM, H₂O up to 10 mL) and a total of 2 μg of DNA was added. The solution was then transferred in a cuvette of Amaxa electroporation. The cuvette was then inserted in the Nucleofection Magic Buffer, and the selected programme (U23) was applied.

An amount of 500 μL MEM α+20% FBS was added to the solution, and half solution was delicately transferred into a well of a 12-well plate containing MEM α+20% FBS (final volume: 1 mL per well). The other half solution was transferred in a well of another 12-well plate. The cells were incubated at 37° C. and at 5% of CO2 for 24 hours. The procedure was repeated for each condition, for a total of 8 conditions: 5HSE-Hsp70±HSF1; 15HSE-Hsp70±HSF1; 45HSE-Hsp70±HSF1; negative control±HSF1.

### Heat Shock

After 24 hours of incubation, on day 1 one of the two plates containing the 8 conditions was incubated at 43° C. and 5% CO₂ for 2 hours with the purpose of activating the Hsp70 promoter. The plate was then incubated again at 37° C. and 5% CO2 for 24 hours.

### FACS Analysis

After 24 hours, on day 2 the cells were detached by trypsinization, resuspended in 300 μl of sorting buffer (1:1 PBS/MEM α+20% FBS, 2% BSA, 1% Penicillin/Streptomycin) and loaded in tubes with filter. The samples were analysed with FACS Aria IIIU.

List of sequences

SEQ ID No. 3-Nucleic acid sequence of HSPA6
promoter
GGTCGGG TGAGGCGCAAA AGGATAAAAA GCCGGTGGAA
GCGGAGCTGA GCAGATCCGA GCCGGGCTGG CTGC
SEQ ID No.2-Nucleic acid sequence of 15HSE-Hsp70
promoter
AAGCTT CTCGAG GAGG CGAAA CTTCT GGAAT ATTCC CGAAA
CGAAA CTTCT GGAAT ATTCC CGAAA CGAAA CTTCT GGAAT
ATTCC CGAAA GTT GGTCGGG TGAGGCGCAAA AGGATAAAAA
GCCGGTGGAA GCGGAGCTGA GCAGATCCGA GCCGGGCTGG CTGC
AAGCTT GAATTC
SEQ ID No. 3-Nucleic acid sequence of 45HSE-Hsp70
promoter
AAGCTT CTCGAG GAGG CGAAA CTTCT GGAAT ATTCC CGAAA
CGAAA CTTCT GGAAT ATTCC CGAAA CGAAA CTTCT GGAAT
ATTCC CGAAA CGAAA CTTCT GGAAT ATTCC CGAAA CGAAA
CTTCT GGAAT ATTCC CGAAA CGAAA CTTCT GGAAT ATTCC
CGAAA CGAAA CTTCT GGAAT ATTCC CGAAA CGAAA CTTCT
GGAAT ATTCC CGAAA CGAAA CTTCT GGAAT ATTCC CGAAA
GTT GGTCGGG TGAGGCGCAAA AGGATAAAAA 15 GCCGGTGGAA
GCGGAGCTGA GCAGATCCGA GCCGGGCTGG CTGC AAGCTT
GAATTC
SEQ ID No. 4-HSE sequence
CGAAA CTTCT GGAAT ATTCC CGAAA

1. An expression vector comprising (i) a temperature-responsive promoter operably linked to (ii) at least one nucleic acid sequence encoding for a regulatory factor of myogenesis and/or at least one nucleic acid sequence encoding for a regulatory factor of adipogenesis.

2. The vector according to claim 1, wherein, said promoter comprises a plurality of temperature-responsive elements (heat shock elements, HSE), preferably wherein said plurality of HSE comprises a number equal to 15 HSE or a number equal to 45 HSE.

3. The vector according to claim 1, wherein said promoter further comprises a nucleic acid sequence of the HSPA6 promoter, operably linked to said plurality of HSE, preferably wherein said nucleic acid sequence of the HSPA6 promoter has the nucleotide sequence SEQ ID No. 1.

4. The vector according to claim 1, wherein said promoter has the nucleotide sequence SEQ ID No. 2 or SEQ ID No. 3.

5. The vector according to claim 1, wherein said regulatory factor of myogenesis is selected from MyoD, myogenin, MYF-5 and MRF4, preferably wherein said regulatory factor of myogenesis is MyoD.

6. The vector according to claim 1, wherein said regulatory factor of adipogenesis is selected from PPARγ and C/EBPβ, preferably wherein said vector comprises a first nucleic acid sequence encoding for the regulatory factor of adipogenesis PPARγ and a second nucleic acid sequence encoding for the regulatory factor of adipogenesis C/EBPβ.

7. The vector according to claim 1, selected from:
a) a vector comprising (i) a promoter comprising 45 temperature-responsive elements, operably linked to (ii) a first nucleic acid sequence encoding the regulatory factor of myogenesis MyoD and to (iii) a second nucleic acid sequence encoding a chromatin remodelling agent (CRA); wherein said second nucleic acid sequence is operably linked to said first nucleic acid sequence by means of an IRES sequence; preferably wherein said promoter has the nucleotide sequence SEQ ID No. 3; and
b) a vector comprising (i) a promoter comprising 15 temperature-responsive elements, operably linked to (ii) a first nucleic acid sequence encoding for the regulatory factor of adipogenesis PPARγ and to (iii) a second nucleic acid sequence encoding for the regulatory factor of adipogenesis C/EBPβ; preferably wherein said promoter has the nucleotide sequence SEQ ID No. 2.

8. The vector according to claim 1, wherein said vector is a viral vector, preferably wherein said vector is a lentiviral vector.

9. Engineered cells having myogenic and/or adipogenic capacity, comprising an expression vector capable of inducing the differentiation of said cells in muscle and/or adipose cells in response to a temperature change.

10. Engineered cells having myogenic and/or adipogenic capacity, comprising the expression vector according to claim 1.

11. The cells according to claim 10, wherein said cells are pluripotent stem cells and/or mesenchymal stem cells, preferably are induced pluripotent stem cells (iPSC).

12. The cells according to claim 10, wherein said cells comprise the following vectors:
a) a vector comprising (i) a promoter comprising 45 temperature-responsive elements, operably linked to (ii) a first nucleic acid sequence encoding for the regulatory factor of myogenesis MyoD and to (iii) a second nucleic acid sequence encoding a chromatin remodelling agent (CRA); wherein said second nucleic acid sequence is operably linked to said first nucleic acid sequence by means of an IRES sequence; preferably wherein said promoter has the nucleotide sequence SEQ ID No. 3;
b) a vector comprising (i) a promoter comprising 15 temperature-responsive elements, operably linked to (ii) a first nucleic acid sequence encoding for the regulatory factor of adipogenesis PPARγ and to (iii) a second nucleic acid sequence encoding for the regulatory factor of adipogenesis C/EBPβ; preferably wherein said promoter has the nucleotide sequence SEQ ID No. 2; and
c) a vector comprising at least one nucleic acid sequence encoding for HSF1 under control of the constitutive promoter CAG.

13. An in vitro method for the preparation of a muscle and/or adipose tissue, preferably cultured meat, comprising the following steps:
a) culturing the cells as defined in claim 10 at a temperature suitable for the proliferation of said cells;
b) increasing the cultivation temperature so as to promote the differentiation of said cells in muscle and/or adipose cells;
c) culturing the differentiated cells so as to obtain said tissue, and optionally
d) preparing an edible product with the tissue obtained in said step c).

14. The in vitro method according to claim 13, wherein said step c) is carried out in at least one second reactor comprising a culture medium, which at least one second reactor is operably linked to means for the regulation of temperature in said second reactor, preferably wherein said means for the regulation of temperature comprises a plurality of resistors.

15. The in vitro method according to claim 13, wherein said step b) is carried out at a temperature comprised between 2° and 45° C., preferably wherein in said step b) and/or c), the temperature is gradually increased during the differentiation process.

16. A kit comprising at least one vector according to claim 1.

17. A kit comprising the cells of claim 10.
